(19) United States
(12) Patent Application Publication
(10) Pub. No.: US 2004/0093658 A1
(43) Pub. Date: May 20, 2004

(54) Maintenance-free cap

(76) Inventor: Jovan Jackson, San Diego, CA (US)

Correspondence Address: The Law Office of Antony M. Novom, P.O. Box 221126, San Diego, CA, 92192-1126, US

(21) Appl. No.: 10/298,861

(22) Filed: Nov. 18, 2002

(51) Int. Cl. A61F 9/00

(52) U.S. Cl. 2/195.1; 2/12; 2/200.1; 40/329

(57) ABSTRACT

A maintenance-free cap that is stylish and appeals to the fashion conscious user. Such a cap will be made from a thin plastic sheet or band, have a fitment layer disposed on its inner surface, and a comfort layer disposed over the fitment layer. The plastic sheet will resist the formation of stress marks and fractures when subject to bending or crushing. Interchangeable logos may be affixed to any surface of the plastic sheet or band.

### BACKGROUND OF THE INVENTION

1. Field of the Invention

The present invention relates generally to caps. In particular, the invention relates to maintenance-free baseball cap or visor that can be adorned with buttons or logos, such as stickers bearing embroidered and/or printed designs, which are detachable and interchangeable.

2. Description of Related Art

There are many styles of caps. A common style is called a baseball cap. While this description is not exact in describing a cap configuration, in general, it describes a cap having a fairly large visor and a full crown.

Baseball caps are now high fashion. They became street fashion when rap artists adopted this sign of youth. The fashion crowd hastily added the cap to its uniform of T-shirts and sneakers.

Part of the baseball cap's appeal is that they are unisex and they are relatively inexpensive. Fifty percent of the caps sold are premiums or promotional in nature and advertise everything from seed and feed companies to fast food and amusement parks.

One company, New Era Cap Company of Derby, N.Y., makes several million caps including the official caps of the majority of professional sports teams of the MLB, NBA, NHL, and college teams.

A typical cap has six pieces in the crown (each with an eyelet for ventilation) and a visor three inches long and seven and a half inches wide. Hollywood's version, as worn by Tom Cruise in “Top Gun” has a somewhat longer bill for dramatic effect.

The design of the baseball cap has remained basically unchanged over the last century. The bill is slightly longer now than it was before World War II, and in the past thirty years the crown has been stiffened and raised a bit in front, which reduces the skull cap effect.

One disadvantage associated with the common baseball cap is its tendency to get dirty. As with all items made of cloth or fabric, merely wearing them outdoors or perspiring while wearing them will result in the item becoming soiled. Further, when the typical baseball cap gets wet, it tends to deform and/or shrink from its original configuration. Addressing these issues, manufacturers and retailers have advised customers of several ways to maintain the traditional cap.

Of the various suggested maintenance procedures, most recommend drying a wet cap on the owners head to maintain proper fitment, otherwise, the cap will lose it's shape. With regard to a soiled cap, the recommended procedure is having the cap dry cleaned or, in the alternative, taking a wet washcloth with a little detergent to the headband of the cap. While wiping down a dirty headband may temporarily clean it, the procedure hardly addresses the dirty exterior of the cap. Further, constantly dry cleaning a favorite cap quickly becomes a costly proposition.

Some have strived to overcome the problem of how to clean a cap without damaging it. For example, with respect to baseball caps, it is known to provide a cage-like structure that surrounds the cap and maintains its shape while washing in a conventional clothes washer. While these washing devices may allow the owner to wash a cap, oftentimes the result is a clean cap that does not fit quite like it did before. These cap washing devices are usually available in a single size fits all without taking into account varying diameter sizes. Thus, one is often left with a clean hat that no longer fits.

U.S. Pat. No. 1,190,427 discloses a cap constructed entirely of fabric so that it may be repeatedly washed and easily kept in a sanitary condition. It is made of a plurality of segmental-shaped pieces of fabric, with an annular sweat band and a visor composed of a plurality of layers of canvas, fiber cloth or similar stiff washable fabric. However, it is known that over time, repeated washing of any fabric will lead to degradation of the product.

Over the years since the first use of the baseball cap, plastic has become widely used in most of today's products. Plastic is now used as the main material in most protective headgear. For example, professional sports helmets, such as those used for football, construction hard hats, and recreational protective gear, such as bicycle helmets. Further thin plastic novelty hats have become popular items for festive occasions, such as New Years and birthday parties.

U.S. Pat. No. 4,551,860 discloses a hat or cap that is made of a decoratively rigid or semi-rigid material, such as metal or plastic. The airy mesh-type head covering enables air to circulate within the hat while still enabling a stylish coiffure to be fully visible while the hat is being worn. The patent teaches a method of making such a hat and addresses the ability to clean it by dunking the hat in a lake or the ocean without damage thereto. However such a rigid hat will resist folding and/or collapsing for easy transport.

U.S. Pat. No. 5,826,278 discloses a one-piece, lightweight, molded plastic hat that comprises a thin, molded plastic sheet forming a hat wall dome having a thickness of less than 3/16 inch. The sheet at the dome forms a corrugation having a local portion defining a U-shaped cross section (resembling a Stetson hat). However, such novelty hats are usually thin enough to fold or collapse, the plastic used is typically brittle and can permanently lose its shape or even crack if folded or collapsed.

Society today demands technological advances that make life easier. Such new advances include single use disposable cutting boards, disposable food storage containers, and small, light-weight folding chairs and bicycles. However, few have addressed the issue that arises when someone decides not to continue wearing his hat.

U.S. Pat. No. 6,349,415 discloses a baseball cap designed to be collapsible in order to be compact enough to conveniently fit in a shirt pocket. This cap has a loop membrane forming the collapsible area with a material covering the entire cap. The material is preferably fabric-based, but other suitable materials may be used. U.S. Pat. No. 5,754,983 discloses a sport cap having a thick-walled neoprene foam rubber visor. The cap can be transported while crushed together in any form desired, with the visor always returning to its original form, once the cap is placed on the head. However, such collapsible hats having an internal framework typically are not rugged enough to withstand everyday use and can be uncomfortable to the user. Further, a foam rubber sports cap departs from the appeal of the fashion crowd.

Of other concern to the fashion crowd is the decorative aspect of the baseball cap. Since the earliest use of the baseball cap, it has been decorated both in overall color and with a logo or insignia identifying the team for which the user plays. Nowadays, most baseball caps sport some kind of logo or design on the font face of the cap that typically includes symbols, slogans or decorative designs. Baseball caps having these kinds of logos are particularly popular as they allow the wearer to express their allegiance to sporting teams, companies, etc., and also to express their individual tastes in apparel.

Unfortunately, most logos are either sewn or printed onto the fabric of the baseball cap which limits the cap to displaying only a single logo. If the wearer wishes to display a different logo on his baseball cap, he must then purchase another cap bearing the desired logo. However, most wearers of baseball caps do not want to purchase a different cap for each logo they wish to wear. Consequently, there has been a need in the prior art for a baseball cap that has interchangeable logos.

U.S. Pat. No. 4,611,355 discloses a baseball cap where the front face and the visor, are covered with pile material which permits different logos displaying insignias and the like, having hook fastener material as backing, to be positioned on the front face of the cap to thereby present a different appearance for the baseball cap. The hook fastener material is the same type of hook fastener and pile material sold under the trademark “VELCRO.” Similarly, U.S. Pat. No. 5,070,545 discloses a cap assembly which has a front face covered by pile material allowing logos with different designs and insignias and hook fastener material as backing, to be positioned on the front face of the cap. U.S. Pat. No. 5,509,144 discloses a baseball cap configured to permit different logos to be interchangeably positioned on the cap. The crown portion is comprised of a plurality of panels sewed onto a rib structure and a hat band. The logos are equipped with hook fastener material which permits them to be detachably mounted on the pile material of the crown portion. A piece of buckram material is positioned on the inside of the crown portion to maintain the front face in a shape where it is substantially perpendicular to the visor. However, repeatedly changing logos on these caps which use hook and pile fastening material can result in either the pile material stretching or even ripping, or the hook fastener material eventually degrading or becoming embedded with dirt, and no longer being usable.

Accordingly, there is a need in the art for an improved cap which maintains its shape while after getting wet, is easy to clean, can be folded or collapsed for transport, and is able to be decorated with a plethora of permanent or removable logos.

### SUMMARY OF THE INVENTION

It is an object of the present invention to overcome limitations in the known art. It is an object of the present invention to provide a cap or visor that may be used without worry of deformation due to water, dirt, or physical manipulation.

These and other objects and advantages of the present invention are achieved in a first implementation of a maintenance-free cap comprising a thin, molded plastic sheet of uniform thickness forming a crown portion having an inner side and an outer side, an outwardly extending bill portion having an top side and a bottom side, a fitment layer disposed over said inner side of said crown portion, and a comfort layer disposed over said fitment layer. In one embodiment, the sheet has a thickness ranging from about 1/8 inch to about 1/4 inch. In another embodiment, the bill portion is thicker than the crown portion. In this and in other embodiments, the plastic sheet has one or more textures selected from the group consisting of smooth, glossy, rough, and matte. In this and in other embodiments, the plastic resists the formation of stress marks and fractures when subject to bending or crushing. In another embodiment, the plastic sheet is translucent. In this embodiment, the plastic sheet comprises UVA and UVB blocking characteristics.

In another embodiment, the fitment layer comprises foam rubber and may or may not be removable. In this and in other embodiments, the comfort layer comprises one or more materials selected from the group consisting of cotton, nylon, rayon, and spandex, and may or may not be removable.

In another embodiment, the crown portion comprises a plurality of evenly spaced ports. In yet another embodiment, the bill portion comprises an anti-glare layer disposed over the bottom side. In yet another embodiment, there are one or more interchangeable logos affixed to the plastic. In this embodiment, the logos comprise foam rubber.

Alternatively, in a second implementation of the invention, a circumferential portion of the crown portion is removed, thereby forming a visor. In this implementation the maintenance-free visor comprises a thin, molded plastic band forming a partial crown portion having an inner side and an outer side, and an outwardly extending bill portion having an top side and a bottom side, the sheet having a substantially uniform thickness; a fitment layer disposed over the inner side of the partial crown portion; and a comfort layer disposed over the fitment layer. In an embodiment of this implementation, the band is not of uniform height. In another embodiment, the sheet has a thickness ranging from about 1/8 inch to about 1/4 inch. In this and other embodiments, the bill portion is thicker than the partial crown portion

In another embodiment, the fitment layer comprises foam rubber and may or may not be removable. In yet another embodiment, the comfort layer comprises one or more materials selected from the group consisting of cotton, nylon, rayon, and spandex. In this and in other embodiments, the comfort layer may or may not be removable.

In another embodiment, the partial crown portion comprises a plurality of evenly spaced ports. In this and in other embodiments, the plastic band has one or more textures selected from the group consisting of smooth, glossy, rough, and matte. In another embodiment, the plastic resists the formation of stress marks and fractures when subject to bending or crushing.

In another embodiment, the bill portion comprises an anti-glare layer disposed over the bottom side. In yet another embodiment, the plastic band is translucent. In this embodiment, the plastic band comprises UVA and UVB blocking characteristics. In yet another embodiment, there are one or more interchangeable logos affixed to the plastic. In this embodiment, the logos comprise foam rubber.

### BRIEF DESCRIPTION OF DRAWING FIGURES

Further objects of the present invention together with additional features contributing thereto and advantages accruing therefrom will be apparent from the following description of the preferred embodiments of the invention which are shown in the accompanying drawings with like reference numerals indicating like components throughout, wherein:

FIG. 1 is a front perspective view of a first implementation of the present invention in the form of a baseball cap;

FIG. 2 is a back perspective view of the first implementation of the present invention

FIG. 3 is a side-perspective view of the first implementation of the invention with a portion cut away for illustrative purposes only;

FIG. 4 is a side perspective view of the first implementation of the invention with the entire crown portion cut away for illustrative purposes only;

FIG. 5 is a bottom perspective view of the first implementation;

FIG. 6 is a side perspective view of the second implementation of the present invention in the form of a visor; and

FIG. 7 is a top perspective view of the second implementation of the present invention.

### DETAILED DESCRIPTION OF PREFERRED EMBODIMENTS

The present invention provides a maintenance-free cap that is stylish and appeals to the fashion conscious user. Such a cap will be made from a thin plastic, have a fitment layer disposed on its inner surface, and a comfort layer disposed over the fitment layer. As used herein, the term “cap” refers to any head covering with a visor and no brim. Such caps contemplated by the invention include the traditional baseball style cap, a modified visor developed therefrom, and the like.

Implementation I

Referring now to FIG. 1, there is shown a front perspective view of a first implementation of the present invention in the form of a baseball cap 100. The baseball cap 100 comprises a crown portion 105 made of a durable, flexible material such as plastic, and an outwardly extending bill portion 110 of like material. As used herein, the term “plastic” refers to any of numerous organic, synthetic, or processed materials that are mostly thermoplastic or thermosetting polymers of high molecular weight that can be molded, cast, extruded, drawn, or laminated into objects, films or filaments. The plastic used in the invention may be of any color, have light-transmitting characteristics ranging from 0% to 100% transmission, and glow or fluoresce. In an embodiment of the invention, the plastic is able to block UVA and UVB transmission.

In one embodiment of the first implementation of the invention, the crown portion 105 and bill portion 110 are of equal thickness. In another embodiment, the bill portion 110 is substantially thicker.

In a preferred embodiment of the invention, the crown portion 105 and bill portion 110 are formed from a singular sheet of plastic ranging in thickness from about 1/8 inch to about 1/4 inch. However, in another embodiment, it is contemplated that the crown portion 105 and bill portion 110 are formed from separate sheets of plastic and either permanently or removably attached to one another. Both the crown portion 105 and bill portion 110 may be formed with any of a number of desired textures, ranging from a high gloss (resembling the traditional batting helmet) to a dull, rougher matte finish (resembling a cloth cap). In a preferred embodiment, the plastic will resist stress marks and fractures when subject to bending or crushing.

In another embodiment of the invention, the crown portion 105 is formed with a plurality of ports 115 to allow heat transfer from the user's head to the atmosphere. In a preferred embodiment of the first implementation of the invention, there are formed six evenly spaced ports 115 circumferentially located about the upper half of the crown portion 105. It should be understood that this invention contemplates any configuration of ports formed within the crown portion 105.

With reference now to FIG. 2, in another embodiment of the invention, the crown portion 105 comprises an adjusting means 130 for proper fitment on a plurality of heads of different circumference. The adjusting means comprises at least two straps 125 formed into the lower region of the crown portion. The straps 125 having a securing means (not shown) for removable attachment to each other. Such securing means include, but are not limited to hook and fastener material, snaps, buttons, adhesives and the like.

Referring back to FIG. 1, there is shown in the first implementation of the present invention, an embodiment having one or more removable logos or insignia 120 attached to the crown portion 105. The logos 120 comprise any insignia, badge, emblem, decoration, regalia, alphanumeric symbol, or artistic rendition of any of the above that will adhere, either permanently or removably, to the plastic used in the embodiments of the invention. By way of example, and not limitation, the logos of the invention include adhesively-backed embroidered patches, stickers, transfers, paintings and drawings. Logos may be further enhanced with the addition of light-emitting diodes (LEDs), light bulbs, glitter, sequins, or the like. As will be appreciated, any surface of the crown portion 105 or the bill portion 110 can be covered with the logos 120 without departing from the scope of the present invention.

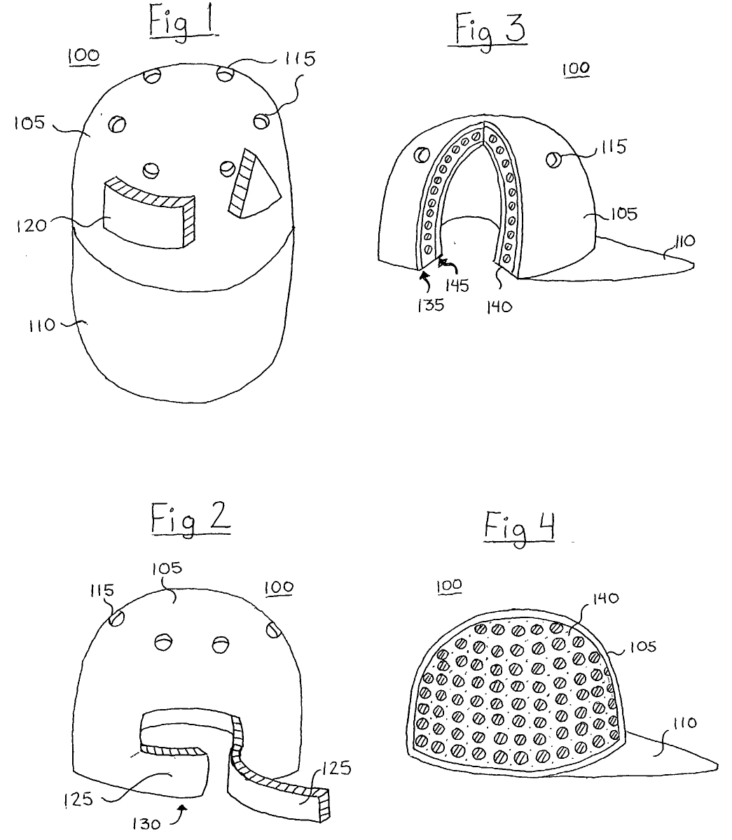

FIG. 3 is a side-perspective view of the first embodiment of the invention with a portion cut away for illustrative purposes only. In an embodiment of the invention, disposed over the inner surface 135 of crown portion 105 is fitment layer 140. Fitment layer 140 comprises any substance that reduces the circumference of crown portion 105 so as to better fit the user's head, and is either permanently or removably attached by an attachment means to the inner surface 135 of the crown portion 105. Such attachment means include, but are not limited to, hook and fastener material, snaps, buttons, adhesives and the like.

In a preferred embodiment, the fitment layer comprises natural or synthetic foam or foam rubber. As used herein, the term “foam” refers to any material in a lightweight cellular form resulting from the introduction of gas bubbles during manufacture. The term “foam rubber” refers to any spongy rubber of fine texture made from latex by foaming before vulcanization. The fitment layer may be of any thickness, although it is contemplated that a thinner layer is better for heat dissipation.

Referring now to FIG. 4, there is shown a side perspective view of an embodiment of the invention with crown portion 105 cut away for illustrative purposes only. Fitment layer 140 can be seen in it's entirety.

Referring back to FIG. 3, there is shown comfort layer 145 disposed over the fitment layer 140. Comfort layer 145 comprises any natural or synthetic textile fibers attached either permanently or removably to the surface of the fitment layer 140 to serve as a barrier between the user's head and the crown portion of the invention. It should be understood that all combinations of crown portion with fitment layer and comfort layer, crown portion with fitment layer, and crown portion with comfort layer are contemplated by the invention. In an embodiment of the invention, comfort layer 145 comprises any breathable cotton or terry cloth sheet. In a preferred embodiment, comfort layer 145 comprises any of various elastic textile fibers made chiefly of polyurethane, such as spandex.

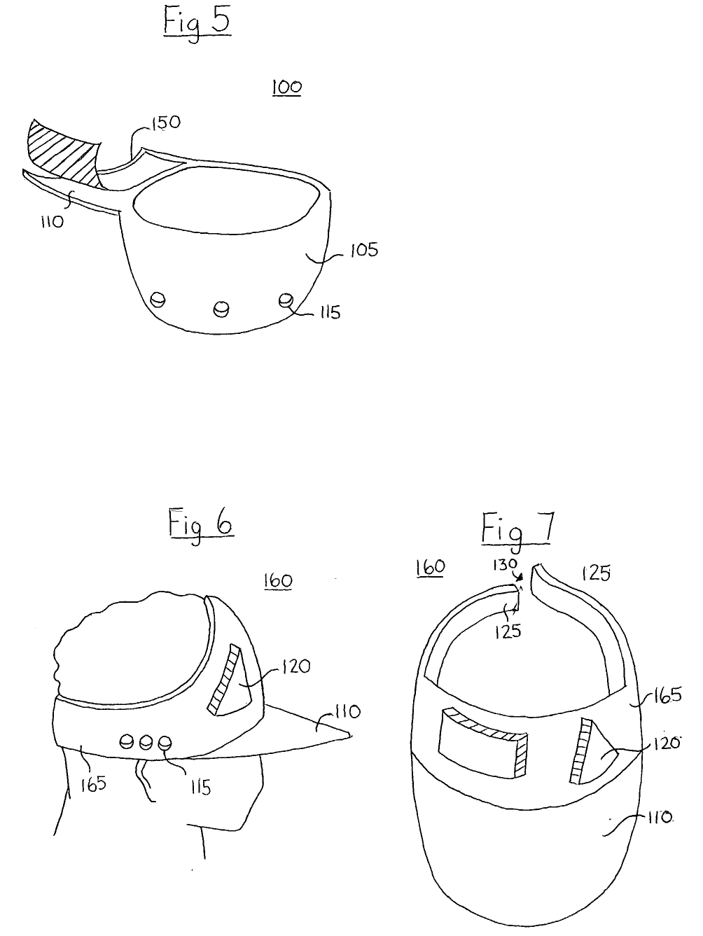

Referring now to FIG. 5, there is shown a bottom perspective view of an embodiment of the first implementation of the invention. Disposed over the bottom side of bill portion 110 is an anti-glare layer 150. Anti-glare layer comprises any natural or synthetic textile fibers attached either permanently or removably to the bottom side of the bill portion 110 to inhibit reflection of light rays that may impair the user's vision.

Implementation II

In FIG. 6, there is shown a side perspective view of a second implementation of the present invention in the form of a visor 160. The visor 160 comprises a partial crown portion 165 made of a durable, flexible material such as plastic, and an outwardly extending bill portion 110 of like material.

As above, in another embodiment of the second implementation of the invention, the partial crown portion 165 and bill portion 110 are of equal thickness. In yet another embodiment, the bill portion 110 is substantially thicker.

In a preferred embodiment of the invention, the partial crown portion 165 and bill portion 110 are formed from a singular sheet of plastic. In another embodiment, the partial crown portion is formed from a plastic sheet in the shape of a band. In yet another embodiment, the band is not of uniform height, thereby providing a visor replicating the look of a baseball cap with the top portion removed therefrom.

It is contemplated that embodiments of the invention may comprise a partial crown portion 165 and a bill portion 110 that are formed from separate sheets of plastic and either permanently or removably attached to one another. Both the partial crown portion 165 and bill portion 110 may be formed with any of a number of desired textures, ranging from a high gloss (resembling the traditional batting helmet) to a dull, rougher finish (resembling a cloth cap). In a preferred embodiment, the plastic will resist stress marks and fractures when subject to bending or crushing.

In another embodiment of the invention, the partial crown portion 165 is formed with a plurality of ports 115 to allow heat transfer from the user's head to the atmosphere. It should be understood that this invention contemplates any configuration of ports formed within partial crown portion 165.

An embodiment of the visor 160 is shown in FIG. 5 having one or more removable logos or insignia 120 attached to the partial crown portion 165. As will be appreciated, any surface of the partial crown portion 165 or the bill portion 110 can be covered with the logos 120 without departing from the scope of the present invention.

With reference now to FIG. 7, in yet another embodiment of the invention, the partial crown portion 165 comprises an adjusting means 130 for proper fitment on a plurality of heads of different circumference. The adjusting means comprises at least two straps 125 formed into the lower region of the crown portion. The straps 125 having a securing means (not shown) for removable attachment to each other. Such securing means include, but are not limited to hook and fastener material, snaps, buttons, adhesives and the like.

As in implementation I, in an embodiment of implementation II, disposed over the inner surface 135 of partial crown portion 165 is fitment layer 140 (see FIG. 3). Fitment layer 140 comprises any substance that reduces the circumference of partial crown portion 165 so as to better fit the user's head, and is either permanently or removably attached by an attachment means to the inner surface 135 of the crown portion 105. Such attachment means include, but are not limited to, hook and fastener material, snaps, buttons, adhesives and the like. The fitment layer may be of any thickness, although it is contemplated that a thinner layer is better for heat dissipation.

Further, an embodiment of Implementation II comprises comfort layer 145 disposed over the fitment layer 140 (see FIG. 3). Comfort layer 145 comprises any natural or synthetic textile fibers attached either permanently or removably to the surface of the fitment layer 140 to serve as a barrier between the user's head and the crown portion of the invention. It should be understood that all combinations of partial crown portion with fitment layer and comfort layer, partial crown portion with fitment layer, and partial crown portion with comfort layer are contemplated by the invention. In an embodiment of the invention, comfort layer 145 comprises any breathable cotton or terry cloth sheet. In a preferred embodiment, comfort layer 145 comprises any of various elastic textile fibers made chiefly of polyurethane, such as spandex. As discussed above, the bottom side of bill portion 110 may be covered with an anti-glare layer 150 to protect the user's eyes from reflected light rays (see FIG. 5).

It is contemplated that both implementations of the invention may be used with or without a slipcover. As used herein, the term “slipcover” refers to any cover that may be slipped off and on the invention. The slipcover may be formed from any material, including, but not limited to, natural or synthetic textile fibers, plastics, and metals. The slipcover serves to both decorate and protect the implementations of the invention.

While this invention has been described in detail with reference to certain preferred embodiments, it should be appreciated that the present invention is not limited to those precise embodiments. Rather, in view of the present disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Having thus described the invention with the details and particularity required by the patent laws, what is claimed and desired protected by Letters Patent is set forth in the appended claims.

What is claimed is:

1. A maintenance-free cap comprising:
a thin, molded plastic sheet forming a crown portion having an inner side and an outer side, and an outwardly extending bill portion having an top side and a bottom side, said sheet having a substantially uniform thickness;
a fitment layer disposed over said inner side of said crown portion; and
a comfort layer disposed over said fitment layer.

2. The cap according to claim 1, wherein the sheet has a thickness ranging from about 1/8 inch to about 1/4 inch.

3. The cap according to claim 1, wherein the bill portion is thicker than the crown portion.

4. The cap according to claim 1, wherein the fitment layer comprises foam rubber.

5. The cap according to claim 1, wherein the fitment layer is removable.

6. The cap according to claim 1, wherein the comfort layer comprises one or more materials selected from the group consisting of cotton, nylon, rayon, and spandex.

7. The cap according to claim 1, wherein the comfort layer is removable.

8. The cap according to claim 1, wherein the crown portion comprises a plurality of evenly spaced ports.

9. The cap according to claim 1, wherein the plastic sheet has one or more textures selected from the group consisting of smooth, glossy, rough, and matte.

10. The cap according to claim 1, wherein the plastic resists the formation of stress marks and fractures when subject to bending or crushing.

11. The cap according to claim 1, wherein the bill portion comprises an anti-glare layer disposed over the bottom side.

12. The cap according to claim 1, wherein the plastic sheet is translucent.

13. The cap according to claim 12, wherein the plastic sheet comprises UVA and UVB blocking characteristics.

14. The cap according to claim 1, wherein a circumferential portion of said crown portion is removed, thereby forming a visor.

15. The cap according to claim 1, further comprising one or more interchangeable logos affixed to said plastic.

16. The cap according to claim 15, wherein said logos comprise foam rubber.

17. A maintenance-free visor comprising:
a thin, molded plastic band forming a partial crown portion having an inner side and an outer side, and an outwardly extending bill portion having an top side and a bottom side, said sheet having a substantially uniform thickness;
a fitment layer disposed over said inner side of said partial crown portion; and
a comfort layer disposed over said fitment layer.

18. The visor according to claim 17, wherein the band is not of uniform height.

19. The visor according to claim 17, wherein the sheet has a thickness ranging from about 1/8 inch to about 1/4 inch.

20. The visor according to claim 17, wherein the bill portion is thicker than the partial crown portion.

21. The visor according to claim 17, wherein the fitment layer comprises foam rubber.

22. The visor according to claim 17, wherein the fitment layer is removable.

23. The visor according to claim 17, wherein the comfort layer comprises one or more materials selected from the group consisting of cotton, nylon, rayon, and spandex.

24. The visor according to claim 17, wherein the comfort layer is removable.

25. The visor according to claim 17, wherein the partial crown portion comprises a plurality of evenly spaced ports.

26. The visor according to claim 17, wherein the plastic band has one or more textures selected from the group consisting of smooth, glossy, rough, and matte.

27. The visor according to claim 17, wherein the plastic resists the formation of stress marks and fractures when subject to bending or crushing.

28. The visor according to claim 17, wherein the bill portion comprises an anti-glare layer disposed over the bottom side.

29. The visor according to claim 17, wherein the plastic band is translucent.

30. The visor according to claim 29, wherein the plastic band comprises UVA and UVB blocking characteristics.

31. The visor according to claim 17, further comprising one or more interchangeable logos affixed to said plastic.

32. The visor according to claim 31, wherein said logos comprise foam rubber.
